(19) United States
(12) Patent Application Publication
(10) Pub. No.: US 2020/0069710 A1
(43) Pub. Date: Mar. 5, 2020

(54) COMPOSITIONS FOR TREATMENT OF HYPERGLYCEMIA IN DIABETES MELLITUS PATIENTS

(71) Applicant: PureCircle USA Inc., Chicago, IL (US)

(72) Inventor: Avetik MARKOSYAN, Yerevan (AM)

(21) Appl. No.: 16/605,826

(22) PCT Filed: Apr. 18, 2018
(86) PCT No.: PCT/US2018/028140

§ 371 Date: Oct. 17, 2019

Related U.S. Application Data
(60) Provisional application No. 62/486,657, filed on Apr. 18, 2017.

(51) Int. Cl. A61K 31/704; A61K 9/00; A23L 27/30; A23L 33/105; A23L 2/60

(52) U.S. Cl. CPC A61K 31/704; A61K 9/0053; A23V 2002/00; A23L 33/105; A23L 2/60; A23L 27/36

(57) ABSTRACT

Compositions comprising at least one steviol glycoside are provided for reducing increased blood sugar level (hyperglycemia) in Diabetes mellitus Type 1 and Type 2 patients.

### BACKGROUND OF THE INVENTION

Sugar alternatives are receiving increasing attention due to awareness of many diseases in conjunction with consumption of high-sugar foods and beverages. One of them is Diabetes mellitus, which is believed to affect about 8.5% of world's adult population and the number of cases is still on rise. There are 2 types of Diabetes mellitus—Type 1 and Type 2, wherein Type 2 is the most common and accounts for 90% of all cases.

The most common symptom in Diabetes mellitus patients is the increased level of blood glucose (hyperglycemia) Typically reduction of blood glucose level is achieved by administration of insulin (Type 1) or other anti-diabetic pharmaceuticals (Type 2) from the compound groups including biguanides, thiazolidinediones, secretagogues, sulfonylureas, nonsulfonylurea secretagogues etc.

Another important part of the Diabetes management is a lifestyle change, which includes a diet with low intake of simple sugars such as glucose, sucrose, fructose etc. Sugar alternatives (sweeteners) are widely used to improve organoleptic properties of such diets (mainly by imparting sweet taste).

Many synthetic (artificial) sweeteners such as dulcin, sodium cyclamate and saccharin were banned or restricted in some countries due to concerns on their safety. Therefore non-caloric sweeteners of natural origin are becoming increasingly popular. The sweet herb Stevia rebaudiana produces a number of diterpene glycosides which feature high intensity sweetness and sensory properties superior to those of many other high potency sweeteners.

Stevia rebaudiana is a plant species belonging to the Astracea family, and is native to South America and cultivated now in many parts of the world (Gardana et al., 2003; Koyama et al., 2003; Carakostas et al., 2008). Stevia leaves are naturally sweet, and have been used for sweetening food products for hundreds of years in South America (Soejarto et al., 1982). Extracts of Stevia rebaudiana have been used commercially to sweeten foods in Japan and other Southeast Asian countries for a number of years (Koyama et al., 2003). As a product of nature, the stevia plant leaves contain different sweet tasting components, called steviol glycosides. Reportedly, more than 40 steviol glycosides have been identified that are typically present in the stevia leaf extract (Ceunen and Geuns, 2013; Chaturvedula et al., 2011a,b,c; Chaturvedula and Prakash, 2011a,b; Ohta et al., 2010). Each of these steviol glycosides has its own unique taste profile and sweetness intensity, which can be up to 350 times sweeter than sugar, but all share a similar molecular structure where different sugar moieties are attached to aglycone steviol (an ent-kaurene-type diterpene).

The leaves of the stevia plant contain a mixture of diterpene glycosides in an amount ranging from about 10% to 15% of the total dry weight. These diterpene glycosides are about 30 to 450 times sweeter than sugar. Structurally, the diterpene glycosides are characterized by a common aglycone—steviol, and differ by the presence of carbohydrate residues at positions C13 and C19 of steviol. Typically, on a dry weight basis, the four major steviol glycosides found in the leaves of Stevia are dulcoside A (0.3%), rebaudioside C (0.6-1.0%), rebaudioside A (3.8%) and stevioside (9.1%). Other steviol glycosides include those listed in FDA GRAS Notice (GRN) No. 619 (filed 2 Feb. 2016) which is incorporated herein in its entirety as reference. Non limiting examples of other steviol glycosides include rebaudiosides B, D, E, F, M, N, O, steviolbioside, rubusoside etc.

To date the use of steviol glycosides has been limited only to sweetener and flavor ingredients in various consumables including foods and beverages.

On the other hand, there remains a need to develop orally ingestible compositions containing natural compounds which will provide similar pharmacological effect (antihyperglycemic) effect as many pharmaceuticals used in Diabetes mellitus treatment.

### SUMMARY OF THE INVENTION

The present invention relates generally to compositions comprising at least one steviol glycoside and consumables comprising said compositions, as well as methods for using such compositions and consumables for the treatment of patients with Diabetes mellitus Type 1 and Type 2.

Non limiting examples of steviol glycosides of present invention include rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, steviolbioside, rubusoside, steviol glycosides listed in FDA GRAS Notice (GRN) No. 619, glycosylated steviol glycosides, alpha-glucosylated steviol glycosides (GSGs), fructosylated steviol glycosides, galactosylated steviol glycosides, as well as other steviol glycosides, found in Stevia rebaudiana plant or prepared by chemical, enzymatic synthesis or by fermentation of recombinant microorganisms, and combinations thereof, wherein said combinations comprise 2 to 100 different steviol glycosides molecules, and wherein the % w/w ratio of each glycoside to the total % w/w content of all steviol glycosides is in the range from 0.0000001% to 99.9999999%, more preferably equal to about 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%.

### DETAILED DESCRIPTION

The chemical structures of certain steviol glycosides of present invention are provided in FDA GRAS Notice (GRN) No. 619 (filed 2 Feb. 2016), while others are known to those skilled in art from literature.

The steviol glycosides of the present invention are either isolated from Stevia plants or produced otherwise such as fermentation by recombinant microorganisms, plants, and biotransformation by enzymes.

One embodiment of present invention is a liquid or solid composition, for reducing increased blood sugar level (hyperglycemia) in Diabetes mellitus Type 1 and Type 2 patients, comprising at least one steviol glycoside, wherein the composition comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

Another embodiment of present invention is an orally ingestible liquid or solid consumable, for reducing increased blood sugar level (hyperglycemia) in Diabetes mellitus Type 1 and Type 2 patients, comprising at least one steviol glycoside composition, wherein the orally ingestible liquid or solid consumable comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

Yet another embodiment of present invention is a method of reducing increased blood sugar level (hyperglycemia) in Diabetes mellitus Type 1 and Type 2 patients, by oral ingestion of a liquid or solid composition comprising at least one steviol glycoside, wherein the composition comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

Another embodiment of present invention is a method of reducing increased blood sugar level (hyperglycemia) in Diabetes mellitus Type 1 and Type 2 patients, by oral ingestion of a liquid or solid consumable comprising at least one steviol glycoside composition, wherein the liquid or solid consumable comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

Non-limiting examples of orally ingestible consumables include: drugs, pharmaceutical preparations, cosmetics, including but not limited to toothpaste, mouthwash, cough syrup, chewable tablets, lozenges, vitamin preparations, and the like, food and beverage products including but not limited to carbonated soft drinks, cola flavored carbonated soft drinks, fruit flavored carbonated soft drinks, berry flavored carbonated soft drinks, ready to drink beverages, energy drinks, isotonic drinks, low-calorie drinks, zero-calorie drinks, sports drinks, teas, fruit and vegetable juices, juice drinks, dairy drinks, yoghurt drinks, alcohol beverages, powdered beverages, bakery products, cookies, biscuits, baking mixes, cereals, confectioneries, candies, chocolate, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables.

The steviol glycosides compositions of present invention can be used “as-is” or in combination with other sweeteners, flavors and food ingredients.

Non-limiting examples of sweeteners include, Luo Han Guo extract, mogrosides, glycosylated mogrosides, high-fructose corn syrup, corn syrup, invert sugar, fructooligosaccharides, inulin, inulooligosaccharides, coupling sugar, maltooligosaccharides, maltodextrins, dextrins, limited dextrins, corn syrup solids, glucose, maltose, sucrose, lactose, allulose, tagatose, aspartame, saccharin, sucralose, sugar alcohols and mixtures thereof.

Non-limiting examples of flavors include cola, lemon, lime, orange, grapefruit, banana, grape, apple, pear, pineapple, bitter almond, cinnamon, sugar, cotton candy, vanilla flavors, glycosylated steviol glycosides, NSF01, NSF02, NSF03 NSF04, NSF05 and mixtures thereof.

Non-limiting examples of other food ingredients include flavors, acidulants, organic and amino acids, coloring agents, bulking agents, modified starches, gums, texturizers, preservatives, antioxidants, vitamins, emulsifiers, stabilisers, thickeners, gelling agents, caffeine, theanine, theobromine and mixtures thereof.

It is to be understood that the foregoing description and specific embodiments shown herein are merely illustrative of the best mode of the invention and the principles thereof, and that modifications and additions may be easily made by those skilled in the art without departing for the spirit and scope of the invention, which is therefore understood to be limited only by the scope of the appended claims.

1. A liquid or solid composition, for reducing increased blood sugar level (hyperglycemia) in Diabetes mellitus Type 1 and Type 2 patients, comprising at least one steviol glycoside,
wherein the composition comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

2. An orally ingestible liquid or solid consumable for reducing increased blood sugar level (hyperglycemia) in Diabetes mellitus Type 1 and Type 2 patients, comprising at least one steviol glycoside composition, wherein the orally ingestible liquid or solid consumable comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

3. A method of reducing increased blood sugar level (hyperglycemia) in Diabetes mellitus Type 1 and Type 2 patients by oral ingestion of a liquid or solid composition comprising at least one steviol glycoside,
wherein the composition comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.

4. A method of reducing increased blood sugar level (hyperglycemia) in Diabetes mellitus Type 1 and Type 2 patients by oral ingestion of a liquid or solid consumable comprising at least one steviol glycoside composition,
wherein the liquid or solid consumable comprises 0.0000001% to 99.9999999% (w/w on dried basis), more preferably 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%; 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% steviol glycosides and combinations thereof.
